(19) United States
(12) Patent Application Publication
(10) Pub. No.: US 2008/0033515 A1
(43) Pub. Date: Feb. 7, 2008

(54) DEVICE FOR OPTICALLY STIMULATING COLLAGEN FORMATION IN TISSUE

(76) Inventor: Andrew F. Kurtz, Macedon, NY (US)

Correspondence Address: Susan L. Parulski;Patent Legal Staff, Carestream Health, Inc., 150 Verona Street, Rochester, NY, 14608, US

(21) Appl. No.: 11/856,177

(22) Filed: Sep. 17, 2007

Related U.S. Application Data
(62) Division of application No. 11/185,650, filed on Jul. 20, 2005.

(51) Int. Cl. A61B 18/18

(52) U.S. Cl. 607/88

(57) ABSTRACT

A polarization based medical device for optically stimulating the formation of collagen in tissue comprises a light source for providing a beam of light. A polarizer polarizes the beam of light. A first beam shaping optics directs the polarized beam of light to a spatial light modulator. A second beam shaping optics directs the polarized beam from the spatial light modulator to an area of interest within the tissue. A spatially controlled pattern of polarized light is directed onto the tissue, thereby affecting the orientation of formation of collagen within the tissue.

### CROSS REFERENCE TO RELATED APPLICATIONS

This is a Divisional application of commonly-assigned co-pending U.S. patent application Ser. No. 11/185,650, filed Jul. 20, 2005 entitled “A DEVICE FOR OPTICALLY STIMULATING COLLAGEN FORMATION IN TISSUE” by Kurtz, the disclosures of which are incorporated herein.

### FIELD OF THE INVENTION

The invention relates generally to a light therapy medical device for influencing the formation of collagen in skin during wound healing. The device could also be used for other medical purposes where the formation and orientation of collagen in tissue can be stimulated and modified.

### BACKGROUND OF THE INVENTION

In general, the healing of wounds, burns, and other injuries is an uncertain endeavor. The clinician cannot be certain about the condition of the tissue being treated, the efficacy of treatments, and whether further treatments or a change in treatments is appropriate. As a particular example, many chronic wounds, such as pressure ulcers or venous stasis ulcers linger for months or even years, often despite the various treatments being applied. These wounds are particularly intractable for a variety of reasons, with age, nutrition, diabetes, infection, marginalized immune systems, and other factors all contributing to the ongoing difficulties in healing. In most cases, such wounds are chronic because the wound healing is stalled relative to one or more aspects of the process. In such circumstances, it is not unusual for the clinician to be unsure about the status of the wounded tissue, at what point in wound healing the tissue is held up, and what new treatment modality should be applied.

As wounds heal, they normally progress through a sequence of overlapping interactive phases, starting with coagulation and progressing through inflammation, proliferation (which includes granulation, angiogenesis, and epithelialization), and remodeling. Success in wound healing is very much dependent on the rebuilding of the extra-cellular matrix (ECM) and granulation tissue, which is initially dependent on fibroblasts. Fibroblasts migrate into the wound site, and begin to build the ECM by depositing a protein called fibronectin. The fibronectin is deposited with some directionality, mirroring the axis of the fibroblasts. The fibroblasts then produce collagen, with the collagen deposition generally aligned to the fibronectin pattern. Over time, fibronectin is replaced by Type III collagen and ultimately by Type I collagen. As the wound contracts, and is subsequently remodeled and influenced by stresses from neighboring tissues, the collagen becomes increasingly organized. Even late in the remodeling phase, which can end six months to a year post injury, collagen in a scar will be replaced and rearranged as the wound attempts to regain its original function.

As the deposition and re-organization of collagen is a key to wound healing, a variety of medical technologies have been developed to influence it. For example, collagen welds and collagen scaffolds or grafts, can be applied to a wound site, to provide the foundational structure for healing. For example, Novartis provides a product called Apligraf, which is a bi-layered tissue therapy, using a lower dermal layer combining bovine Type I collagen and human fibroblasts, which produce additional matrix proteins, with an upper epidermal layer formed using human keratinocytes (epidermal cells

As a potential alternative, external light therapy has been shown to be effective in treating various medical conditions, including the treatment of wounds, burns, and other skin surface (or near skin surface) ailments, as well as other conditions such as seasonal affective disorder (SAD), psoriasis, acne, and hyperbilirubinemia common in newborn infants. In the 1960's and 1970's researchers in Eastern Europe undertook the initial studies that launched modern light therapy. One such pioneer was Endre Mester (Semmelweiss Hospital, Budapest, Hungary), who in 1966 published the first scientific report on the stimulatory effects of non-thermal ruby laser light (694 nm) exposure on the skin of rats. Professor Mester found that a specific range of exposure conditions stimulated cell growth and wound healing, while lesser doses were ineffective and larger doses were inhibitory. In the late 1960's, Professor Mester reported the use of laser light to treat non-healing wounds and ulcers in diabetic patients. Mester's 70% success rate in treating these wounds lead to the development of the science of what he called “laser biostimulation.”

Presently, there are over 30 companies world wide that are offering light therapy devices for a variety of treatment applications. These devices vary considerably, with a range of wavelengths, power levels, modulation frequencies, and design features being available. In many instances, the exposure device is a handheld probe, comprising a multitude of light emitters; that can be directed at the patient during treatment. The light emitters, which typically are laser diodes, light emitting diodes (LEDs), or combinations thereof, usually provide light in the red-IR (˜600-1200 nm) spectrum, because the tissue penetration is best at those wavelengths. In general, both laser light and incoherent (LED) light seem to provide therapeutic benefit, although some have suggested that lasers may be more efficacious. Light therapy is covered by a variety of terms, including low-level-laser therapy (LLLT), low-energy-photon therapy (LEPT), and low-intensity-light therapy (LILT). Despite the emphasis on “low,” many of the products marketed today output relatively high power levels, of up to 1-2 optical watts. Companies that presently offer light therapy devices include Thor Laser (United Kingdom), Omega Laser Systems (United Kingdom), MedX Health (Canada), Quantum Devices (United States), and Lumen Photon Therapy (United States).

Many different examples of light therapy and PDT devices are known in the patent art. Early examples include U.S. Pat. No. 4,316,467 (Muckerheide) and U.S. Pat. No. 4,672,969 (Dew). The most common device design, which comprises a hand held probe, comprising at least one light emitter, but typically dozens or even 100 emitters, that is attached to a separate drive controller, is described in numerous patents, including U.S. Pat. Nos. 4,930,504 (Diamantapolous et al.); 5,259,380 (Mendes et al.); 5,464,436 (Smith); 5,634,711 (Kennedy et al.); 5,660,461 (Ignatius et al.); 5,766,233 (Thiberg); and 6,238,424 (Thiberg).

The light therapy devices that are commercially available today are disadvantaged in that the clinician does not know either the optical dosage delivered (light into the tissue) or the effective dosage delivered (light-tissue interaction). In part, the uncertainty is because many participants are not well educated in optics, and do not know how to measure light properly. However, the uncertainty is also because the science of light therapy is complicated. The leading theory for light therapy describes a process in which cytochrome oxidase (and other bio-chemicals), absorb incident light energy thus generating free electrons, which are then transferred within the mitochondrial electron transport chain to produce biochemicals such as adenosine triphosphate (ATP). ATP is then used in various cellular processes (including the synthesis of proteins and RNA). Additionally, various cell types (fibroblasts, epithelial cells, macrophages, mast cells, etc.) can apparently be stimulated for various effects, with these effects possibly occurring over hours, days, or even weeks.

Despite the various uncertainties concerning the science and efficacy of light therapy, there is considerable effort in the field to develop improved methods and devices, with a portion of this effort directed in ways that could benefit wound healing in particular. As one factor determining the progress of wound healing is the formation of granulation tissue, which is in turn, dependent on the formation of a collagen network, progress in light therapy that effects collagen is worth consideration. One particular example is the Gentle Waves light therapy device, which was developed by Light Bioscience LLC of Virginia Beach, Va., which is a non-thermal, non-ablative technology using low intensity light-emitting diodes (LEDs) at specially calibrated energies to reduce the visible signs of aging and sun-damaged skin. A related patent, U.S. Pat. No. 6,663,659 (McDaniel) describes using LED based light therapy, with the light dosage (wavelength, intensity, and pulse conditions) optimized to the action spectra of various cell types, such as fibroblasts. Based on data, such as the fibroblast action spectra (see FIGS. 5-12), treatment protocols were developed to treat fine lines and wrinkles, as then discussed in examples 1-3 therein. The same researcher has also disclosed a second light therapy technique, described in U.S. Pat. No. 6,676,655 (McDaniel), which employs pulsed femtosecond yellow laser light (590 nm) to induce stimulatory effects in fibroblasts. In both patents, the intended effect of the light therapy is to then stimulate the formation and remodeling of collagen fibers and bundles in order to improve scar tissues and skin damage. Pending U.S. Patent Application Publication No. 2005/0149150 (McDaniel), is another variant of this type of light therapy, where yellow light and infrared light are used in combination to stimulate fibroblasts and collagen formation.

Numerous other studies, aside from the work of McDaniel, have examined the effects of light therapy on fibroblasts, although relatively few of these studies have been in-vivo instead of in-vitro. The experimental work published in “Effects of Low-Intensity Polarized Visible Laser Radiation on Skin Burns: A Light Microscopy Study” in the Journal of Clinical Laser Medicine and Surgery, Vol. 22, pp. 59-66, 2004, by Martha Simoes Ribiero et al., is of particular interest, as it involves in-vivo light therapy effecting collagen production and wound healing. In this study, rats were deliberately burned on the back, near the spine, and then treated with red polarized laser light therapy, where the progress of healing was examined relative to the polarization orientation of the incident light. In particular, a HeNe laser (632 nm, 10 mW) provided a beam that was expanded and pre-polarized to uniformly expose a wound, illuminating a 1 cm² area with 6 mW. The rats were exposed every third day post wounding for 3 minutes per exposure (1 J/cm²). It was found that the wounds that were irradiated with polarized light (polarization either parallel or perpendicular to the spinal column) healed faster than the control non-irradiated wounds. Moreover, the wounds treated with light polarized parallel to the spine healed the fastest and exhibited both an enhanced proliferation of fibroblasts and the most pronounced organization of the collagen fibrils. While this work is provocative, the article does not discuss why the polarization orientation, relative to the native tissue, would accelerate wound healing. The article further does not discuss how application of polarized light could be applied beneficially to the healing of complex wounds, such as chronic wounds, like pressure ulcers. Finally, and most importantly, this article does not suggest the design of a practical device or devices that could be useful in providing polarized light therapy treatment to wounds generally, and in particular, to large complex wounds such as chronic wounds like pressure ulcers.

### SUMMARY OF THE INVENTION

Briefly, according to one aspect of the present invention a polarization based medical device for optically stimulating the formation of collagen in tissue comprises a light source for providing a beam of light. A polarizer polarizes the beam of light. A first beam shaping optics directs the polarized beam of light to a spatial light modulator. A second beam shaping optics directs the polarized beam from the spatial light modulator to an area of interest within the tissue. A spatially controlled pattern of polarized light can be directed onto the tissue, thereby affecting the orientation of formation of collagen within the tissue.

The invention and its objects and advantages will become more apparent in the detailed description of the preferred embodiment presented below.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 is a cross-sectional view of the epidermal and dermal layers of the skin.

FIG. 2 is a histological cross-sectional picture of a tissue sample, showing a fibroblast and collagen structures.

FIGS. 3a and 3b are two histological cross-sectional picture showing collagen structures in skin.

FIG. 4 is an illustration of Langer's cleavage lines.

FIG. 5 is a picture of a pressure ulcer.

FIG. 6 is a diagrammatic illustration of a chronic wound.

FIGS. 7a and 7b depict perspective views of spatially variant polarized light therapy devices of the present invention.

FIG. 8 depicts a cross-sectional view of a spatially variant polarized light therapy of the present invention.

### DETAILED DESCRIPTION OF THE INVENTION

Reference is made to commonly-assigned copending U.S. patent application Ser. No. 11/087,183, filed Mar. 23, 2005 entitled WOUND HEALING MONITORING AND TREATMENT by Kurtz; and U.S. patent application Ser. No. 11/087,300, filed Mar. 23, 2005 entitled LIGHT THERAPY DEVICE by Olson et al., the disclosures of which are incorporated herein.

The present invention will be directed in particular to elements forming part of, or in cooperation more directly with the apparatus in accordance with the present invention. It is to be understood that elements not specifically shown or described may take various forms well known to those skilled in the art.

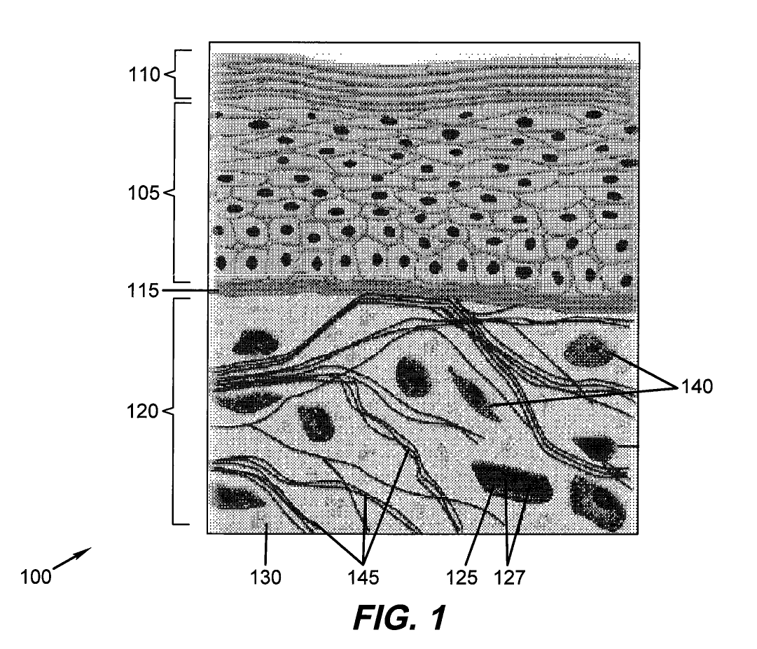

The present invention can be best appreciated within the context of the biology of normal, wounded, and healed skin, and in particular, with respect to the function of fibroblasts and collagen. Accordingly, FIG. 1 depicts the cross-sectional composition of skin. Skin 100 (or the integument) covers the entire external surface of the human body and consists of two mutually dependent layers, the epidermis 105 and the dermis, which rest on a fatty subcutaneous layer, the panniculus adiposus (not shown). The epidermis 105, which is the outer layer of skin, is made up of epithelial cells (also known as squamous cells or keratinocytes), basal cells, and melanocytes. The outermost layer of the epidermis 105 comprises layers of dead epithelial cells 110. The basal cells are responsible for producing the epithelial cells, while the melanocytes produce pigments (melanin) that give skin its color. Below the epidermis 105 is the basement membrane 115 (also known as the basal lamina), which helps attach the epidermis 105 to the reticular dermis 120. The basal lamina 115 actually comprises several layers, and includes proteoglycans and glycoproteins as well as Type IV collagen. The innermost layer of the basal lamina 115 includes several types of fibrils, including collagen Type III and Type VII fibrils, which help anchor to the dermis. The dermis comprises several layers, including the papillary dermis (not shown) and the reticular dermis 120, which is the primary dermal layer. The papillary dermis is composed of fine networks of Types I and III collagen, elastic fibers, ground substance, capillaries and fibroblasts. The reticular dermis 120 contains thick collagen bundles (thicker than the papillary dermis), which are arranged in layers parallel to the surface of the skin. In FIG. 1, the reticular dermis 120 is shown, with constituent blood capillaries 125 with transiting red blood cells 127, fibroblasts 140, collagen fiber bundles 145, and proteoglycans 130. Proteoglycans 130 are large molecules that attract and hold water, thereby providing cushioning and support. The reticular dermis 120 also contains other structures (not shown), such as elastin, sebaceous glands, sweat glands, hair follicles, and a small number of nerve and muscle cells.

The dermal skin layers vary with body location. For example, skin is quite thin on the eyelids, but is much thicker on the back and the soles of the feet. The epidermis ranges in thickness from ˜30 microns to ˜1 mm, while the dermis (papillary and reticular) ranges between ˜300 microns and ˜3 mm in thickness. The collagen structure in skin also varies with location, as will be discussed subsequently.

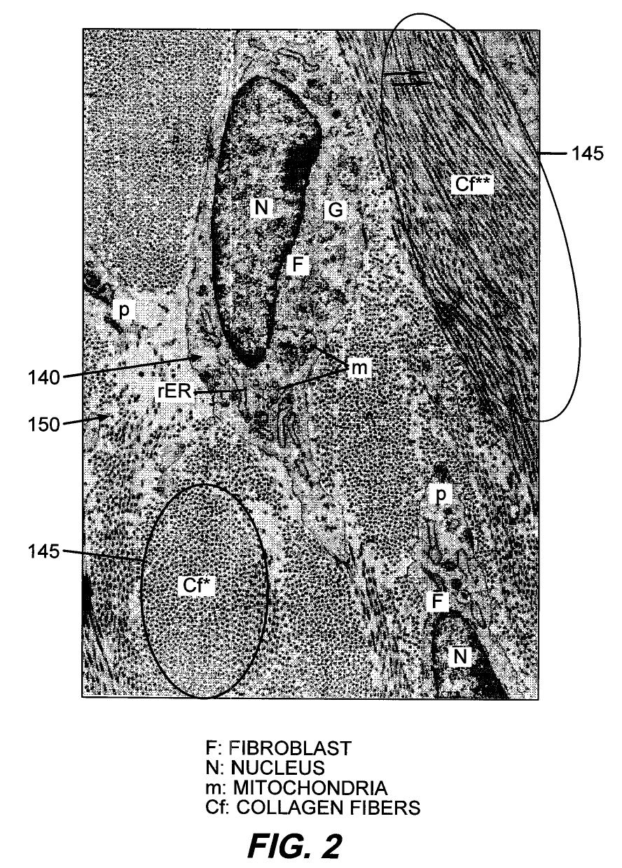

Fibroblasts create many of the components of the connective tissue in the reticular dermis, including the elastin, fibronectin, and collagen, which are all complex fibrous proteins. Collagen actually comprises long bundles or strands, composed of innumerable individual collagen fibrils. A fibroblast 140, is depicted in a histology image in FIG. 2, with at least four collagen fiber bundles 145, comprising numerous individual collagen fibrils 150, seen both in cross section and in plane within the image. Fibroblasts synthesize collagen (both Type I and Type III), in a process beginning with procollagen, which is polymerized outside the fibroblasts to form tropocollagen, which in turn is formed into collagen fibrils and collagen bundles. The collagen fibril segments are ˜25-50 microns in length and ˜10-200 nm in diameter (depending on type). These fibril segments fuse linearly and laterally (crosslink) to form longer, thicker, biomechanically competent collagen fibrils 150 within collagen fiber bundles 145, which can be 200 microns in length. Smaller collagen bundles can be 0.5-10 microns in diameter, although thicker bundles, particularly in the reticular dermis, can be ˜100 microns in diameter. Notably, Type III collagen fibers are generally thinner than the Type I fibers. Fibroblasts within a well-formed collagen network tend to be elongated themselves.

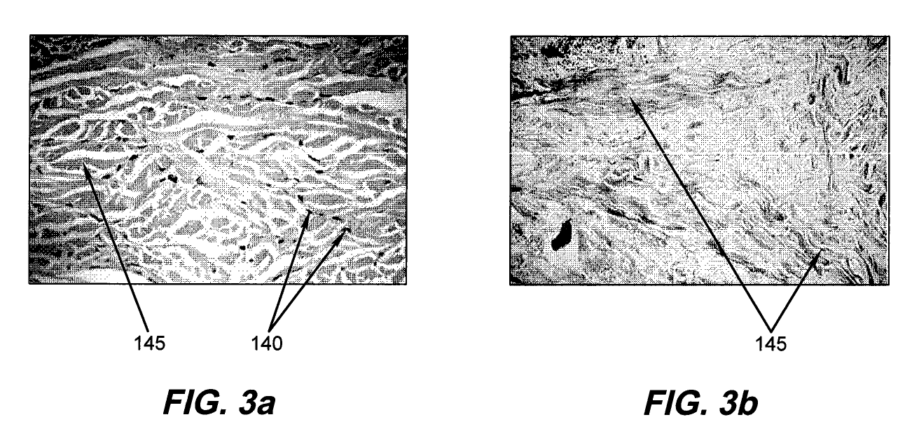

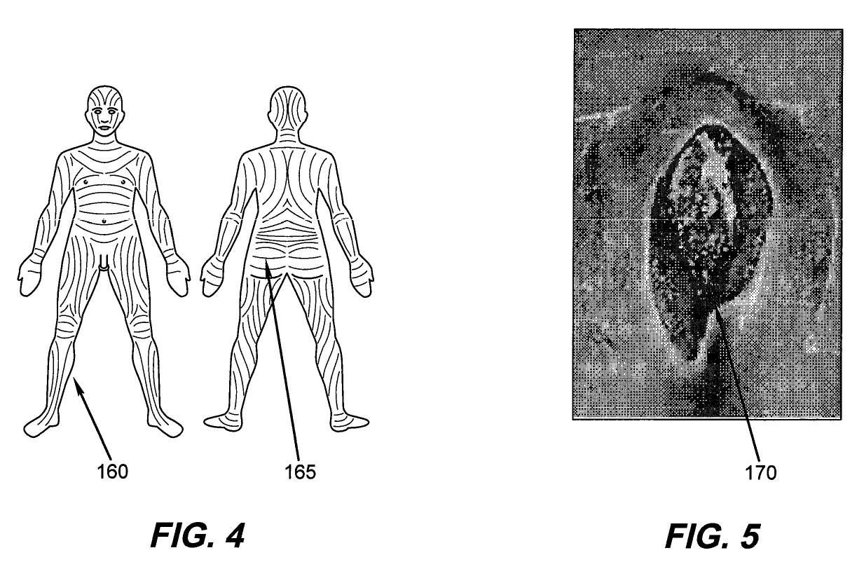

The most structured collagen formations are found in bones and tendons. The collagen structures in tendons, ligaments, and vocal cords, which are termed “dense regular” and have collagen fibers in parallel alignment, are structured to handle stresses and transmit forces along their length. By comparison, the collagen structures in skin (see FIGS. 3a and 3b), in which the collagen fibrils and bundles are less organized and somewhat wavy or convoluted, are termed “dense irregular.” Although some researchers have described the collagen structures in skin to be random or haphazard (see FIGS. 3a and 3b), there is both local and macro patterning. Human dermal tissue (skin) is compliant and adapts to pressures from all directions. The collagen network, which is multi-directional and multi-layered (in depth relative to the surface), is an interwoven mesh generally parallel to the surface of the skin, which gives skin its toughness and adaptability. However, there is a pre-dominant direction to the orientation of the fiber bundles in a given location. In areas, such as the abdomen, where Langer's lines are the most prominent, as much as 35% of the collagen may be aligned with the direction of pre-dominance. As shown in FIG. 4, Langer's cleavage lines 165 are generally associated with the alignment of collagen bundles deep in the reticular dermis. These lines portray the directional effects of skin across the human body 160, wherein the stress-strain relationships in uniaxial tension show skin to be stiffer along Langer's lines than across the lines. Langer's lines have been shown to correlate within +/−10 degrees to the direction of maximum pre-stress in skin.

As a result, Langer's lines 165 are used as guides in surgery, with incisions preferentially running along the lines rather than cutting obliquely through them. This is because incisions along these lines heal with a minimum of scarring, whereas oblique wounds may be pulled apart or develop thicker scars. Extended interconnecting series of collagen bundles that follow Langer's lines may be several millimeters, or even a centimeter or more in extent. Some common directionality, at least on a local scale of a few hundred microns, is evident in the collagen structures in the skin of FIGS. 3a and 3b. Collagen fibers generally do not often branch and, when branches are found, they usually diverge at an acute angle (see FIG. 1).

Wounds are characterized in several ways; acute wounds are those that heal normally within a few weeks, while chronic wounds are those that linger for months or even years. Wounds that heal by primary union (or primary intention) are wounds that involve a clean incision with no loss of substance. The line of closure fills with clotted blood, and the wound heals within a few weeks. Wounds that heal by secondary union (or secondary intention) involve large tissue defects, with more inflammation and granulation. Granulation tissue is needed to close the defect, and is gradually transformed into stable scar tissue. Such wounds are large open wounds as can occur from trauma, burns, and pressure ulcers. While such a wound may require a prolonged healing time, it is not necessarily chronic. A chronic wound is a wound in which normal healing is not occurring, with progress stalled in one or more of the phases of healing. A variety of factors, including age, poor health and nutrition, diabetes, incontinence, immune deficiency problems, poor circulation, and infection can all cause a wound to become chronic. Typical chronic wounds include pressure ulcers, friction ulcers, and venous stasis ulcers. Chronic wounds are also categorized, according to the National Pressure Ulcer Advisory Panel (NPUAP) relative to the extent of the damage:
Stage 1—has observable alteration of intact skin with changes in one or more of skin temperature, tissue consistency, or sensation (pain, itching). Pro-active treatment of Stage 1 and Pre-Stage 1 (also known as Stage 0) wounds could be beneficial.
Stage 2—involves partial thickness skin loss involving epidermis, dermis, or both. The ulcer is superficial and appears as an abrasion, blister, or shallow crater.
Stage 3—Full thickness skin loss with damage or necrosis of subcutaneous tissue.
Stage 4—Full thickness skin loss with extensive destruction, tissue necrosis, and damage to muscle, bone, or supporting structures (tendon, joint, capsule, etc.). Successful healing of Stage 4 wounds still involve loss of function (muscles and tendons are not restored).
Stage 5—Surgical removal of necrotic tissue usually required, and sometimes amputation. Death usually occurs from sepsis.

Wound healing also progresses through a series of overlapping phases, starting with coagulation (haemostasis), inflammation, proliferation (which includes collagen synthesis, angiogenesis, epithelialization, granulation, and contraction), and remodeling. Haemostasis, or coagulation, is the process by which blood flow is stopped after the initial wounding, and results in a clot, comprising fibrin, fibronectin, and other components, which then act as a provisional matrix for the cellular migration involved in the later healing phases. Many of the processes of proliferation, such as epithelialization and angiogenesis (creation of new blood vessels) require the presence of the extracellular matrix (ECM) in order to be successful. Fibroblasts appear in the wound during that late inflammatory phase (˜3 days post injury), when macrophages release cytokines and growth factors that recruit fibroblasts, keratinocytes and endothelial cells to repair the damaged tissues. The fibroblasts then begin to replace the provisional fibrin/fibronectin matrix with the new ECM. The ECM is largely constructed during the proliferative phase (˜day 3 to ˜2 weeks post injury) by the fibroblasts, which are cells that synthesize fibronectin and collagen. As granulation continues, other cell types, such as epithelial cells, mast cells, endothelial cells (involved in capillaries) migrate into the ECM as part of the healing process.

Fibroblasts initial role in wound healing is to provide fibronectin, which is a glycoprotein that promotes cellular adhesion and migration. Fibronectin weaves itself into thread-like fibrils, with “sticky” attachment sites for cell surfaces, to help connect the cells to one another. There is some directionality to the deposition of fibronectin, which in turn impacts the deposition of the other ECM proteins. Fibroblasts synthesize collagen (both Type I and Type III), beginning with procollagen, which is three polypeptide chains (each chain is over 1400 amino acids long) wound together in a tight triple helix. Procollagen is then extruded from the fibroblast out into the extracellular space. Once exocytosed, these filaments lay disorganized in the wound, still in a gelatinous state. The triple-helical molecule undergoes cleavage at specific terminal sites. The helix is now called a tropocollagen molecule, and tropocollagens spontaneously associate in an overlapping array. The amassing continues as tropocollagen convolves with other tropocollagen molecules to form a collagen fibril. Wound durability, or tensile strength, is dependent on the microscopic welding (cross-linking) that must occur within each filament and from one filament to another. The collagen fibril segments are ˜25-50 microns in length and ˜10-200 nm in diameter (depending on type). The fibril segments fuse linearly and laterally (crosslink) to form longer, thicker, biomechanically competent collagen fibrils 150 within collagen bundles 145. Collagen deposition will align itself to the fibronectin pattern, which in turn mirrors the axis of the fibroblasts. Although the initial collagen deposition may appear somewhat haphazard, the individual collagen fibrils are subsequently reorganized, by cross-linking, into more regularly aligned bundles oriented along the lines of stress in the healing wound, and eventually, at least partially, to the stress lines associated with the surrounding tissue.

Type III collagen is the type that appears in the wound initially, starting at about four days after injury. Collagen becomes the foundation of the wound ECM, and if collagen formation does not occur, the wound will not heal. Myofibroblasts, which are a specialized fibroblast, appear late during the proliferative phase (at ˜5 days), to help contract the wound so that there will be less scarring. Wound contraction helps to further organize the early collagen structures. A ring of these contractile fibroblasts convene near the wound perimeter, forming a “picture frame” that will move inward, decreasing the size of the wound.

As wound healing progresses into the remodeling stage (starting at ˜10 days post injury) the fibroblasts continue to work to build more robust tissue structures. Matrix synthesis and the remodeling phase are initiated concurrently with the development of granulation tissue and continue over prolonged periods of time (˜30-300 days, depending on the injury). As the extracellular matrix matures, fibronectin and hyaluronan (a component of the proteoglycans) are broken down. Over time, fibronectin is replaced by Type III collagen and ultimately by Type I collagen. Type III collagen is fairly quickly replaced by Type I collagen, which constitutes 90% of the total collagen in the body, and forms the major collagen type found in the reticular dermis. As remodeling progresses, towards a goal of having the new ECM match the original and fit with the surrounding tissue, the collagen structure is altered on an ongoing basis, by a process of lysis and synthesis. Collagen degradation is achieved by specific matrix metalloproteinases (MMPs) that are produced by many cells at the wound site, including fibroblasts, granulocytes and macrophages. Gradually, the Type I collagen bundles are deposited with increasing organization, orientation, and size (including diameter), to better align to the surrounding tissues and increase wound tensile strength.

An ideal case of wound healing is one in which there is a complete regeneration of lost or damaged tissue and there is no scar left behind. In the case of a minor acute wound, which heals by primary intention, there will be little or no scarring, and the final tissue will be basically equivalent to the original. In the cases of an acute wound that heals by secondary intention (multiple layers of skin are injured), the healed wound will likely include some portion of scar tissue. Scars start as granulation tissue with large irregular mass of collagen. As with the primary union degree wound, scar remodeling for a secondary union type wound continues, attempting to mimic the surrounding tissue in structure and strength. The amount of scar to be remodeled is inversely related to the return of function. However, typically the fully healed scar has only 70-80% of the strength of the original tissue. In part this is because the collagen bundles never match fully match the original, nor regain the original alignments. Additionally, as adults produce few new elastin fibers during healing, the scar lacks the elasticity and recoil of the original tissue.

In the case of acute wounds, the efforts of the fibroblasts and the myofibroblasts to build the ECM and granulation tissue, and to close the wound, can be exhibited in a “collagen ridge” or “healing ridge,” which is a region surrounding the wound (extending perhaps ˜1 cm on each side) where new collagen synthesis is occurring. During treatment, clinicians often have to locate the collagen ridge by feel (palpitation), in order to assess the wound condition and treatment. However, the collagen ridge may be poorly defined and difficult to locate.

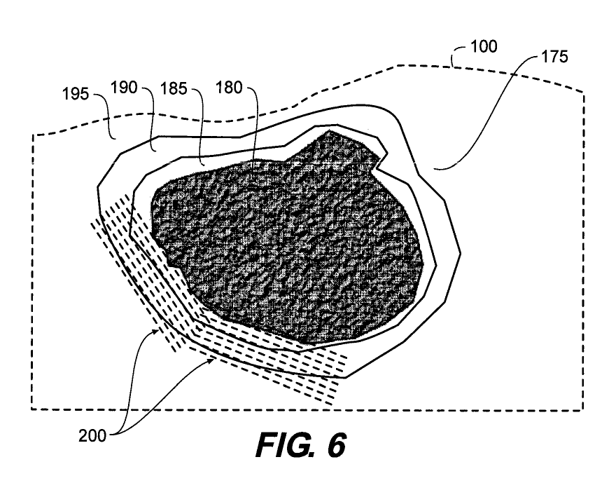

As previously stated there are several types of chronic wounds, including the pressure ulcer (or decubitis ulcers or bed sores), all of which suffer impaired healing. Stage 3 and Stage 4 pressure ulcers (see FIG. 5) are open wounds that can occur whenever prolonged pressure is applied to skin covering bony outcrops of the body. Patients who are bedridden are at risk of developing pressure ulcers. Stage 4 pressure ulcers can form in 8 hours or less, but take months or years to heal. Pressure ulcers 170 are complicated wounds, which can include infection, slough (dead loose yellow tissue), black eschar (dead blackened tissue with a hard crust), hyperkeratosis (a region of hard grayish tissue surrounding the wound), and undermining or tunneling (an area of tissue destruction extending under intact skin). Pressure ulcers may have closed wound edges (epibole), which impedes healing. In such circumstances, the top layers of the epidermis have rolled down to cover lower edge of epidermis, including the basement membrane, so that epithelial cells cannot migrate from wound edges.

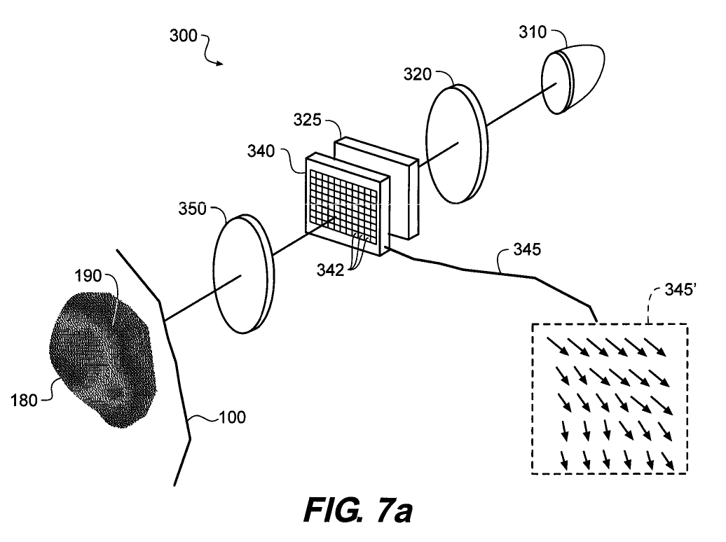

Chronic wounds, such as pressure ulcers, show vastly different tissue structures in the wound bed, as compared to the wound margin (the transition region to normal tissue). The types of tissue structures in and around a wound site also vary with the stage of the wound. This can be understood with reference to FIG. 6, which illustrates a chronic wound 175 with an ulcer bed 180 in the nominal center of the wound, a surrounding ulcer edge 185, a surrounding ulcer margin 190, and adjacent normal tissue 195. The immediate proximal normal tissue 195, which is also referred to as the periwound tissue, can be sensitive and tender. According to the papers “Pressure (decubitus) ulcer: Variation in histopathology—a light and electron microscope study”, by Vande Berg et al., in Human Pathology, Vol. 26, pp. 195-200, 1995, and “Fibroblast Senescence in Pressure Ulcers” by J. S. Vande Berg et al., in Wound Repair and Regeneration, Vol. 6, pp. 8-41, 1998, the ulcer bed includes inflammatory cells (macrophages, neutrophils), as well as oversized, round, vacuolated (having air cavities) fibroblasts. In particular, senescent (aged) fibroblasts were often observed in clusters in the ulcer bed. Senescent fibroblast produce fibronectin that is structurally different from that produced by normal fibroblasts, and as a result, the abnormal fibronectin is unable to bind with collagen. Senescent fibroblasts also lose their ability to produce collagen, but produce high levels of collagenase and low levels of the tissue inhibitors for MMPs. Fibroblast senescence is not uniform in chronic wounds, but varies within areas of the ulcer bed and from patient to patient.

Vande Berg et al. further reports that the ulcer edge, which surrounds the ulcer bed, may have dense collagen bundles, or loose fibrous regions (including collagen) with inflammatory cells and vacuolated fibroblast remnants. Finally, the surrounding ulcer margin was similar to a dense scar tissue, sometimes composed of woven, very dense collagen fiber bundles covered with a thin epidermis, and typically fewer fibroblasts and inflammatory cells than in the ulcer proper. Fibroblasts from the ulcer margin and adjacent normal skin show a continued ability to divide. Not surprisingly, the collagen in healing pressure ulcer tissue is different than that in normal tissue, as there are fewer collagen fibers, but they may be significantly wider and longer than in normal tissue. Not surprisingly, the blood capillaries in and around chronic wounds were few and often occluded (obstructed).

In a somewhat similar aspect to the healing ridge of collagen bordering the edge of an acute wound, the collagen in the ulcer margin and the ulcer edge tends to parallel the wound edges, but is less completely structured. As can now be appreciated, successful collagen formation and remodeling is very important in wound healing, whether the wounds are acute (primary or secondary) or chronic, and whether the wounds are in the inflammatory phase, the proliferative phase, or the remodeling phase, or a combination thereof. Any treatment modality that encourages the formation of a functional collagen network, and thus the further progress of granulation tissue (including angiogenesis) and wound healing has potential value.

According to the previously cited paper by Ribiero et al., the application of polarized light to healing wounds can have a beneficial impact. Notably, Ribiero et al. does not speculate as to the physical mechanism of this effect. It is known however that collagen, and likely other elongated tissue components, such as elastin and fibronectin, are optically birefringent. Optically birefringent materials have multiple indices of refraction, unlike isotropic (homogeneous) media (such as glass) that have a single index of refraction. Light sees varying effective indices of refraction depending on the polarization direction of its electric field when traveling through an anisotropic material. As a result, transiting polarized light will react differently, depending on the relative alignment of the polarization state of the light to the polarization state of the tissue. It could be that the transiting polarized light interacts with electron resonances (as with dipoles) within the procollagen molecules, to thus influence the procollagen orientation and formation within the fibroblasts. This initial procollagen orientation then effects the orientational direction of the resulting tropocollagen and collagen. As a formed collagen network reacts to applied internal and external stresses, the imbedded fibroblasts are likewise stressed.

Notably, it has been described in a paper, Mechanobiology of Force Transduction in Dermal Tissue, by F. Silver et al., in Skin Research and Technology, Vol. 9, pp. 3-23, 2003, that mechanical stress, and changes thereof, affect cytoskeletal re-organization, gene expression, and cell differentiation. In the case of fibroblasts, a modest directional mechanical load causes the fibroblasts to show higher levels of procollagen mRNA, and to adopt a phenotype characterized by induction of connective tissue synthesis and inhibition of matrix degradation. Such fibroblasts tend to organize into a matrix and have longer life spans and enhanced proliferation as compared to fibroblasts that are in a disorganized state. Thus, if the polarized light help establish an initial orientation of the collagen deposition and the related fibroblasts, a virtuous cycle might be established, which further application of polarized light therapy could then enhance.

Based on the previous discussions of wound healing, chronic wounds, and the role and properties of collagen, it can be suggested that therapeutic treatments that encourage collagen formation around wound edges, and in particular parallel to the wound edges, are potentially beneficial. In particular, the polarization light therapy approach that has been experimentally studied by Ribiero et al., could be beneficially applied to wound healing by spatially patterning the application of the polarized light to generally follow the wound edges. This is illustrated in FIG. 6, where two areas of application of polarized light 200 relative to chronic wound 175. Each area of polarized light 200 generally follows the wound edges, and nominally falls on at least the ulcer margin 190, and likely also the ulcer edge 185 and a proximal portion of the neighboring ulcer bed 180. Nominally polarized light therapy could be applied to generally follow around the entire perimeter of the wound site. In practice, a clinician could apply polarized light sequentially around the edges of the wound, until the entire wound perimeter had been treated. Light therapy would assist the formation of a first collagen layer at a first location adjacent to the wound, and then the formation of a second collagen layer at second location adjacent to the wound. The intent would be to encourage collagen formation along the wound edges, and thus the formation of granulation tissue, with capillaries and other tissue structures. Moreover, as the wound changes over the course of time, the polarized light therapy treatment could be applied differently, for example, in accordance with the gradually closing shape of the wound site.

Polarized light therapy might also be applied for wound healing using different wavelengths in a sequential manner. For example, longer IR wavelengths (such as 940 or 1060 nm) that penetrate several millimeters into the tissue might be used first, to encourage collagen formation at deeper tissue depths. Shorter wavelengths (such as 670 nm or 810 nm), which penetrate to lesser depths, could then be used nearer the surface. Such an approach could be applied to assist the tissue in building a more extensive collagen network with collagen layers at different tissue depths. This approach might also be used in wound healing and remodeling, providing multi-directional multi-layered collagen, to rebuild a multi-layered collagen mesh, more similar to that in the normal tissue.

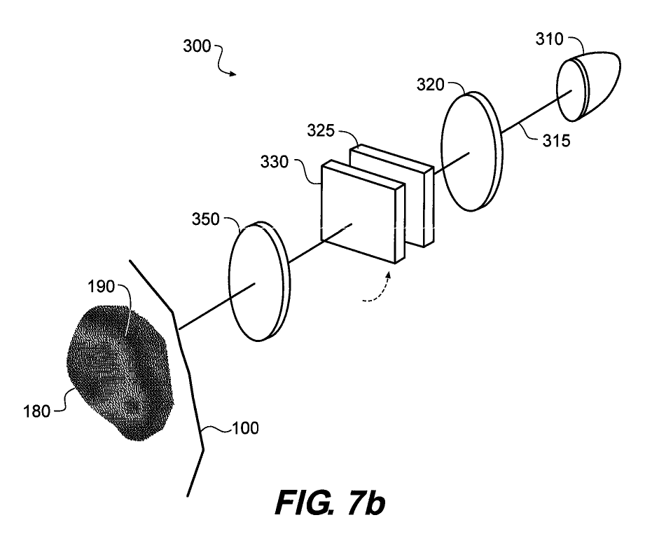

Accordingly, FIG. 7a depicts a first embodiment of the present invention that enables spatially variant polarized light therapy to tissue. In this form, polarization light therapy device 300 includes a light source 310, illumination optics 320, a pre-polarizer 325, a spatial light modulator 340, and beam shaping optics 350, which provides polarized light onto skin (tissue) 100. Spatial light modulator 340 is nominally driven by a drive signal 345, which at least in part, conveys the spatially variant data for polarization signal 345′. Polarization signal 345′ comprises a set of polarization vectors which can directed at skin 100, so that the applied polarized light can follow along the wound edges, such as along the ulcer margin 190.

In greater detail, light source 310 could be a lamp (generally depicted in FIG. 7a), one or more light emitting diodes (LEDs), a laser, one or more laser diodes, or some other light source. Light source 310 preferably emits light (CW or pulsed) in the red and/or near infrared spectra (˜600-1200), where the optical transmission of tissue is the highest. For example, light source 310 could emit red light at 670 nm from one or more LEDs while emitting 830 nm light from one or more laser diodes. Following U.S. Pat. No. 6,663,659, the therapeutic light could also be pulsed yellow (590 nm) light, although the depth of tissue penetration would be reduced compared to red or IR light. Depending on the structure of the light source 310 and the spatial light modulator 340, illumination optics 320 could be designed in a variety of ways. For example, illumination optics 320, which is generally depicted as a simple lens, could actually include one or more beam shaping optical elements known in illumination design. Commonly known optics such as integrating bars or fly's eye integrators could be used to homogenize the illumination if needed. These same components could provide a rectangular beam profile, consistent with the shape and aspect ratio of the modulator array 340. As one example, illumination optics 320 could include a lens to image the output face of an integrating bar (not shown) onto spatial light modulator 340. Alternately, if the light source 310 provides an output light having a small optical invariant (or LaGrange), illumination optics 320 could function to nominally collimate the light, and would then present spatial light modulator 340 with a collimated input light beam, rather than a re-imaged light beam. Likewise, beam shaping optics 350 can be used to image spatial light modulator 340 onto the tissue 100, but if the spatial light modulator 340 is illuminated with a nominally collimated beam, that light could transfer through spatial light modulator 340, and illuminate the tissue directly with patterned polarized light, without having to be imaged. In that case, imaging optics 350 may be unnecessary, and the size of the overall device is reduced. Beam shaping optics 350 could still be used for beam shaping to the tissue (to create convergent light for example), without actually imaging the spatial light modulator 340. However, as light source 310 likely consists of some multitude of discrete emitters (such as LEDs) distributed over some area, the illumination light will not likely be collimated, and the use of beam shaping optics 350 is likely. Beam shaping optics 350, could be, for example, a low cost plastic achromatic lens.

If the light emitted from light source 310 is unpolarized, or if the polarization states are somewhat randomized by the illumination optics and/or the combining of multiple light sources, then a pre-polarizer 325 can be used in the illumination system. As one example, pre-polarizer 325 could be a wire grid polarizer, such as are available from Moxtek Inc. of Orem, Utah. Wire grid polarizers are quite useful, as they provide polarized output light with high transmission. In the case that light source 310 provides polarized output light, pre-polarizer 325 could be used to enhance the polarization if necessary. For efficiency reasons, pre-polarizer 325 would be preferentially aligned to the primary polarization axis of the output light from light source 310. If the light source 310 provides unpolarized output light, then in order to improve light efficiency, it is generally useful to use a polarization converter. For example, pre-polarizer 325 could be incorporated into the illumination optics 320 as a polarization converter. Many examples of polarization converters are known in the art, and are often used in the design of rear projection televisions. Examples include the mounting of a wire grid polarizer on the output face of an integrating bar (see U.S. Pat. No. 6,795,243, (McGettigan et al.), for example), or a fly's eye integrator assembly that includes an array of mini-prism/waveplate pairs (see U.S. Pat. No. 5,978,136 by (Ogawa et al)). A secondary polarizer could follow after the polarization converter, to further enhance the polarization state of the incident light if necessary, such that a wide range of polarization contrasts could be used (˜5:1 to 1,000⁺:1, depending on efficacy). In order to improve the efficiency of the polarization light therapy device 300, a wave plate (not shown) could be provided between pre-polarizer 325 and spatial light modulator 340. While spatial light modulator 340 will be utilized to impart a spatially variant polarization pattern, there could be circumstances, where device operation (such as overall light efficiency) could be improved if an adjustable (by rotation) waveplate (nominally λ/4) was provided prior to spatial light modulator 340, to adjust the polarization orientation supplied to spatial light modulator 340.

Although polarization light therapy device 300 of FIG. 7a somewhat resembles a projection display system, it is indeed different. In a projection display, a polarization analyzer would follow the spatial light modulator 340, so that the rotated polarization states could be turned into intensity variations, as relate to some image, which is then projected onto a target plane. In this case, it is the spatially modified polarization states that are required, and thus polarization light therapy device 300 lacks a post-modulator polarization analyzer.

In the case of the polarization light therapy device 300 of FIG. 7a, spatial light modulator 340 is preferably a broadband transmissive polarization modulator array, such as a liquid crystal display or shutter. For example transmissive LCD micro-displays are widely used today in the construction of rear projection television systems. Such panels are available in numerous sizes and image formats (such as SVGA or HDTV), and are mass produced commodity items. Although other polarization modulators, such as reflective liquid crystal on silicon (LCOS) or PLZT-based devices could be used, a transmissive LCD panel is preferable, because the cost is low and the optical system can have a simple design that follows a single optical axis.

Spatial light modulator 340 nominally comprises an array of pixels 342, which respond to a drive or control signal 345 to then provide polarization signals 345′. Depending of the signal sent to an individual pixel, the polarization orientation of the transmitted light would be rotated by some amount. A group of neighboring pixels would likely all have the same orientations, such that an area of tissue 100 can be illuminated with largely parallel-polarized light. It is anticipated that the polarization vectors would likely change orientation slowly when projected onto the tissue 100, such that a common polarization direction could be held over several mm or several cm in length. Given that expectation, a micro-display LCD panel, such as is used for rear projection television, which typically have pixels with linear dimensions of only ˜8-20 microns, likely provides more resolution than is required. As an alternative, a transmissive LCD panel, such as used for cell phones, digital cameras, or PDA's, with pixel sizes of ˜250-300 microns width, and overall panel sizes of a few square inches, would likely be more appropriate for this application. However, a liquid crystal shutter or panel with even larger pixels, with millimeter or multi-millimeter size dimensions, could be optimal. The modulator selection criteria could be largely economic, as for example; a custom low-resolution panel may cost more than a volume produced cell phone panel. It should be noted that display panels are fabricated with color filter arrays and color pixel addressing, neither of which is needed for this application.

The drive signals 345 for spatial light modulator 340 are nominally provided through a controller (not shown), where a clinician could input the desired polarization light pattern, relative to the tissue to be illuminated. It is desirable for polarization light therapy device 300 to include red (or other visible wavelengths) light emitters, at least so that the clinician knows where the device output is directed, if not also for therapeutic use.

As an alternative, FIG. 7b depicts a simplified version of the device of the present invention. In this system, the function of spatial light modulator 340 is replaced by a direct mechanical rotation of the illuminating polarization states. In FIG. 7b, pre-polarizer 325 is fixed, and a waveplate 330 (nominally ¼ wave) follows in the optical path, and the waveplate is rotated relative to the optical axis 315. Alternately, waveplate 330 could be removed, and polarizer 325 could be rotated directly. For this version of the polarization light therapy device 300, an indicator (not shown) should be provided so that the clinician would know the polarization orientation emerging from polarization light therapy device 300. As compared to the device of FIG. 7a, which used spatial light modulator 340 to provide a spatially variant polarization pattern, with this device, a single polarization state is emitted at a given time, and the clinician provides the spatial variation relative to the tissue 100, by moving polarization light therapy device 300, and either rotating polarization light therapy device 300, or rotating an internal optic such as waveplate 330 or pre-polarizer 325.

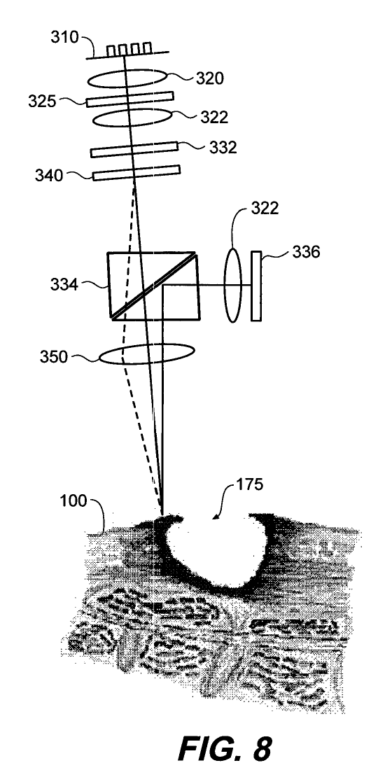

As another alternative, FIG. 8 depicts a more complicated version of a polarization light therapy device 300 of the present invention. In this case, polarization light therapy device 300 is equipped with both a polarization illumination channel and a monitor channel. For compactness, the polarization light therapy device 300 of FIG. 8 is depicted as having the polarization illumination channel and the imaging channel combined by a beamsplitter 334, so that a common optical path is available down to the tissue 100. In particular, the illumination channel comprises light source 310 (depicted as an array of light emitters), illumination optics 320 and 322, pre-polarizer 325, spatial light modulator 340, beamsplitter 334, and beam shaping optics 350. The monitor channel comprises a detector (an area array, such as a CCD or a CMOS device), an optional optic 322 (which could be a field lens, for example), beamsplitter 334, and beam shaping optics 350.

In use, the illumination channel would provide polarized illumination light to tissue 100, as was generally describe previously with the FIG. 7a device. The illumination channel could be operated in a bimodal fashion, with a lower light level for set-up than for therapy. Beamsplitter 334 could be a 50-50 type intensity splitting beamsplitter, or even a dichroic device that reflects 50% of the red or visible light used for pointing, but transmits 100% of the therapeutic infrared light. Beamsplitter 334 could also be an angular sensitive total internal reflection (TIR) prism, but beamsplitter 334 is preferably not a polarization beamsplitter. If such, it would function like a polarization analyzer, and alter the spatially variant polarization states provided by spatial light modulator 340.

Some portion of the incident polarized light will backscatter, and can then be collected by the monitor channel, as images captured by detector 336. The output of detector 336 could be coupled to a display screen (not shown), where the clinician could see an image of the wound site and skin surfaces, showing directly where therapeutic light would be applied. The clinician could then use a user interface to quickly input the desired polarization orientation. To facilitate this, the display screen could be a touch screen panel. Alternately, polarization light therapy device 300 could use internal algorithms to map the captured image of the wound site against therapeutic criteria for applying polarized light, and then send polarization signals 345′ to spatial light modulator 340 accordingly. To capture a good image, beam shaping optics 350 needs to be image conjugate from the tissue 100 to detector 336. Beam shaping optics 350 still need not be image conjugate from the spatial light modulator 340 to the tissue 100, but it could be. Beam shaping optics 350 are then an imaging lens of some sort, and even could be a zooming lens system to facilitate illuminating different sized areas.

The illumination channel may also include a controllable mask 332, so that the illumination to the wound site can be controlled, for example, to prevent therapeutic light from falling on the ulcer bed or on extensive areas of normal tissue. Mask 332 could comprise one or more physical apertures (such as an iris), or a LCD panel with a polarizer, or other means for a spatially controlled light transmission and light blocking. Mask 332 could enable annular illumination, so that the light falls only on the wound edges, and not in the wound bed. Mask 332 could also be included in the systems of FIGS. 7a and 7b, as well as the system of FIG. 8. Alternately, a physical mask could actually be placed in near contact with said tissue, to control where the light is or is not incident onto said tissue. As a result of using a mask, then if the therapies applied to the wound bed are different than those applied to the wound margin, the wound bed does not receive an incorrect therapy.

The devices of the present invention have been described principally with respect to their use in the treatment of chronic wounds, such as pressure ulcers. However, these devices might be used in the treatment of other conditions, such as in the healing of acute wounds, burns, etc. Also, therapeutic light applied to impact collagen orientation could have other uses, such as assisting the remodeling processes that scars undergo during the last stage of wound healing. In that case, scar tissues partially re-organize the collagen network to become more multi-directional, in an attempt to regain as much of the original functionality as possible. Polarized light therapy could assist that process by encouraging collagen formation in new directions within the scar tissues. For example, it could be beneficial to encourage new collagen formation so that the scar collagen was more closely aligned to the Langer's lines orientations of the surrounding or proximal tissues. As one example, polarized light therapy with a sequential, long wavelength then short wavelength, modality, might be used for scar remodeling.

The invention has been described in detail with particular reference to certain preferred embodiments thereof, but it will be understood that variations and modifications can be effected within the scope of the invention.

### PARTS LIST

100 skin
105 epidermis
110 dead epithelial cells
115 basement membrane (basal lamina)
120 reticular dermis
125 blood capillary
127 red blood cells
130 proteoglycans
140 fibroblasts
145 collagen fiber bundles
150 collagen fibrils
160 human body
165 Langer's cleavage lines
170 pressure ulcer
175 chronic wound
180 ulcer bed
185 ulcer edge
190 ulcer margin
195 normal tissue
200 polarized light
300 polarization light therapy device
310 light source
315 optical axis
320 illumination optics
322 optic
325 pre-polarizer
330 waveplate
332 mask
334 beamsplitter
336 detector
340 spatial light modulator
342 pixels
345 drive signal
345′ polarization signal
350 beam shaping optics

1. A polarization based medical device for illuminating tissue with spatially variant polarized light, comprising:
a) an illumination optical system, comprising a light source, having one or more light emitters, illumination beam shaping optics, polarizing optics, and a spatial light modulator for providing spatially variant polarized illumination light;
b) an optical detection system, comprising an optical detector array which receives image light;
c) a beamsplitter for re-directing either said illumination light or said image light such that said spatially variant polarized illumination light and said image light traverse a common optical path between said beamsplitter and said tissue; and
d) an imaging lens for directing said spatially variant polarized illumination light to said tissue and collecting image light from said tissue.

2. A polarization based medical device as in claim 1 wherein said spatial light modulator comprises an array of pixels, which modulate the polarization state of said beam of light in accordance with the applied drive signals.

3. A device as in claim 1 wherein said spatially variant polarized illumination light is applied to affect collagen orientation and formation of one or more pre-existing wounds of said tissue, in order to enhance wound healing.

4. A device as in claim 3 wherein said spatially variant polarized illumination light is applied around the edges of said wounds.

5. A device as in claim 4 wherein said spatially variant polarized illumination light is applied nominally parallel to said edges of said wounds.

6. A device as in claim 3 wherein the directionality of collagen, as described by Langer's lines, relative to proximal tissues to said wounds, is used as a guide to the alignment of said collagen in said proximal tissues, and then as a guide to affecting said collagen orientation and formation.

7. A device as in claim 3 wherein a spatially variable mask is provided to control the locations where the light is and is not applied to said tissue.
